(19) United States
(12) Patent Application Publication
(10) Pub. No.: US 2004/0030585 A1
(43) Pub. Date: Feb. 12, 2004

(54) Method and system for remote image reconstitution and processing and imaging data collectors communicating with the system

(76) Inventor: Aviram Sariel, Ramot Hashavim (IL)

Correspondence Address: G.E. Ehrlich, Anthony Castorina, 2001 Jefferson Davis Highway, Suite 207, Arlington, VA, 22202, US

(21) Appl. No.: 10/399,552

(22) PCT Filed: Oct. 24, 2001
(86) PCT No.: PCT/IL01/00979

(30) Foreign Application Priority Data
Oct. 25, 2000 (IL) 139259

(51) Int. Cl. G06F 17/60

(52) U.S. Cl. 705/3

(57) ABSTRACT

A system for remotely producing a diagnostic image is disclosed. The system includes (a) a server designed and constructed for receiving, via a first communications network, at least one signal acquired by a remote imaging data collector; and (c) an image processor for processing the at least one signal into image data being communicable via a second communications network and presentable via an image presentation device.

### FIELD AND BACKGROUND OF THE INVENTION

The present invention relates to a method and system for remote image reconstitution and processing and to medical imaging data collectors communicating with the system. More particularly, the present invention relates to a system designed and constructed for receiving signals acquired by any one of a plurality of remote diagnostic imaging data collectors, reconstitute the signals collected thereby into images and communicating the resulting images for presentation to a practitioner.

All existing medical diagnostic imaging modalities, such as, but not limited to, x-ray devices, ultrasound devices, computerized tomography is devices, magnetic resonance imaging devices, positron emission tomography devices and nuclear medicine devices include an imaging data collector and an accompanying, dedicated, computational platform which is designed to process the data collected (acquired) by the imaging data collector to complete the process which is known in the art as “image acquisition and reconstitution”. This design results in very expensive machines, mainly because processing of the acquired data into images (image reconstitution) requires the involvement of very sophisticated, high performance and expensive computers and software, and as mentioned, each modality has it's own computer platform integrated therein and/or attached thereto.

The high cost of diagnostic imaging modalities has several consequences, all resulting in lower accessibly of patients to such diagnostic devices and inferior public health as would have been if such devices were less costly.

Among these consequences one can list the following: (i) rural/remote health care facilities not being able to afford such modalities and therefor not being able to offer important diagnostic examinations to their patients; (ii) the cost of examination being high so as to cover the expenses of the health care center for purchasing the diagnostic imaging modality, therefore, the examination itself not being affordable to all patients, resulting, on occasion, in essential examinations not being performed; and (iii) each care center having fewer modalities of each kind, also making such examinations less available to patients. This lower availability of needed examinations to patients results in a delayed discovery of disease and lower prognostic results.

The prior art teaches remote communication of reconstituted images. To this end, see, for example, U.S. Pat. Nos. 6,101,407; 5,976,088; 5,949,491; 5,851,186; and 5,482,043; 5,715,823. However, the prior art fails to teach remote image reconstitution. Thus, at present, there are no medical imaging modalities that lack the computation power required for image reconstitution attached thereto or integrated therewith. On the other hand, there is no system which is designed to remotely communicate with a plurality of imaging data collectors, either sequentially or simultaneously, so as to remotely provide such collectors with a computation power required for image reconstitution.

U.S. Pat. No. 6,050,940 to Braun et al. teaches remote reconstitution of data pertaining to a patient from a plurality of medical sensors. However, this patent refers to the acquisition and remote reconstitution of physiological data such as ECG, EEG, blood pressure, pulse oximetry, etc., yet it fails to describe or suggest remote reconstitution of medical images.

There is thus a widely recognized need for, and it would be highly advantageous to have, a method and system for remote image reconstitution because, by servicing a plurality of imaging data collectors located in a variety of remote locations, such method and system will result in substantially reducing the price of medical imaging systems and consequently their use will be broadened.

### SUMMARY OF THE INVENTION

According to one aspect of the present invention there is provided a system for remotely producing a diagnostic image, the system comprising (a) a server designed and constructed for receiving, via a first communications network, at least one signal acquired by a remote imaging data collector; and (c) an image processor for processing the at least one signal into image data being communicable via a second communications network and presentable via an image presentation device.

According to another aspect of the present invention there is provided a method for remotely producing a diagnostic image, the method comprising the steps of (a) at a source location, using an imaging data collector for acquiring at least one signal; (b) communicating the at least one signal via a first communications network to a remote location; (c) at the remote location, processing the at least one signal into image data; and (d) communicating the image data via a second communications network to the source location or to a third location.

According to yet another aspect of the present invention there is provided a system for providing image reconstitution service, the system comprising (a) a server designed and constructed for receiving, via a first communications network, at least one signal acquired by a remote imaging data collector; and (b) an image processor for processing the at least one signal into image data being communicable via a second communications network and presentable via an image presentation device.

According to still another aspect of the present invention there is provided a method for providing image reconstitution services, the method comprising the steps of (a) receiving at least one signal acquired by a remote imaging collector via a first communications network; and (b) processing the at least one signal into image data, the image data being presentable via an image presentation device; and (c) communicating the image data via a second communications network.

According to further features in preferred embodiments of the invention described below, the first communications network and the second communications network are a single communications network.

According to still further features in the described preferred embodiments the server being further designed and constructed for billing a client.

According to still further features in the described preferred embodiments the server is further designed and constructed for effecting secure credit/debit transactions.

According to still further features in the described preferred embodiments the imaging data collector is selected from the group is consisting of a digital X-ray data collector, an ultrasound data collector, a magnetic resonance data collector, a computerized tomography data collector, a positron emission tomography data collector and a nuclear medicine data collector.

According to still further features in the described preferred embodiments the at least one signal is selected from the group consisting of a raw digital signal, a raw analog signal, and a raw analog signal converted into a digital signal.

According to still further features in the described preferred embodiments the at least one signal is encapsulated, including the packetisation of the at least one signal so as to be self descriptive.

According to still further features in the described preferred embodiments the image data is encapsulated, including the packetisation of the image data so as to be self descriptive.

According to still further features in the described preferred embodiments the image data is represented as a file containing pixel data.

According to still further features in the described preferred embodiments the image data is represented as a video output signal.

According to still further features in the described preferred embodiments the image data is represented as a streaming output signal.

According to still further features in the described preferred embodiments the image data represents a two dimensional image.

According to still further features in the described preferred embodiments the image data represents a three dimensional image.

According to still further features in the described preferred embodiments the system further comprising a data storage device for effecting storage of the image data and retrieval of the image data.

According to still further features in the described preferred embodiments the server is further designed and constructed for automatically communicating the image data back to a default electronic address.

According to still further features in the described preferred embodiments the default electronic address is received by the server, via the first communications network, along with the at least one signal.

According to still further features in the described preferred embodiments the image data is sent, via the second communications network, to an electronic address of a healthcare practitioner, the practitioner being skilled at analyzing and interpreting the image data.

According to still further features in the described preferred embodiments the image data is sent, via the second communications network, to an electronic address of a patient.

According to still further features in the described preferred embodiments the image data is sent, via the second communications network, to any electronic address desirable and authorized by any one of a group comprising the patient, a Health Management Organization a healthcare facility, an insurance company and any combination thereof.

According to still further features in the described preferred embodiments the server is further designed and constructed for automatically analyzing the image data.

According to still further features in the described preferred embodiments the server is further designed and constructed for automatically interpreting the image data.

According to still further features in the described preferred embodiments the image data is retrieved from the data storage device and sent, via the communications network, to an electronic address of a healthcare practitioner, the practitioner being skilled at analyzing and interpreting the image data.

According to still further features in the described preferred embodiments the image data is retrieved from the data storage device and sent, via the communications network, to an electronic address of a patient.

According to still further features in the described preferred embodiments the image data is retrieved from the a data storage device and sent, via the communications network, to any electronic address desirable and authorized by any one of a group comprising the patient, a Health Management Organization, a healthcare facility, an insurance company and any combination thereof.

According to still further features in the described preferred embodiments the server is further designed and constructed for automatically analyzing the retrieved image data.

According to still further features in the described preferred embodiments the server is further designed and constructed for automatically interpreting the retrieved image data.

According to an additional aspect of the present invention there is provided a medical diagnostic device comprising an imaging data collector and lacking image producing capability.

According to further features in preferred embodiments of the invention described below, the device further comprising a communication port, the device being designed and constructed for communicating raw data collected thereby to a remote location.

The present invention successfully addresses the shortcomings of the presently known configurations by providing a

Implementation of the method, device system of the present invention involves performing or completing selected tasks or steps manually, automatically, or a combination thereof. Moreover, according to actual instrumentation and equipment of preferred embodiments of the method, device and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a black box diagram of a system according to the teachings of the present invention and of an image data collector and a presentation with which it communicates;

FIG. 2 is a simplified presentation of an image data collector according to the present invention; and

FIG. 3 is a block diagram featuring the data flow to and from the system of the present invention.

### DESCRIPTION OF THE PREFERRED EMBODIMENTS

The present invention is of a method and system which can be used for remote image reconstitution. The present invention is further of medical imaging data collectors communicating with the system of the present invention, which can be used to collect imagery raw data and communicate the data to the system for image reconstitution. Specifically, the present invention can be used for remotely processing one or more signals containing imagery raw data, which signals are communicated, via a network, from one or more imaging data collectors that are used to examine a patient, into medical diagnostic images. The reconstituted medical diagnostic image may be communicated to the location where the signal acquisition occurs, or to other remote locations, and presented there via a simple image presentation device, for the purpose of analysis and interpretation of the image data by skilled professionals. This architecture of a medical diagnostic system in which a single image data processing computer which serves for image reconstitution communicates with a plurality of data collectors would result in reducing the prices of medical imaging and as a result medical imaging will become more applicable and more widely used.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

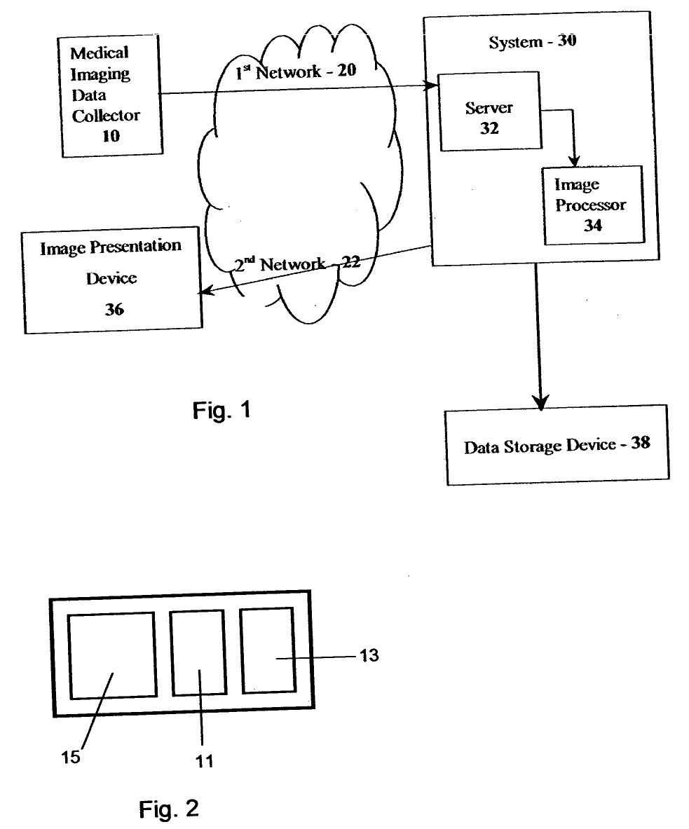

Referring now to the drawings, FIG. 1 illustrates a system for remotely producing a diagnostic image, in accordance with the teachings of the present invention, which system is referred to hereinbelow as system 30.

System 30 includes a server 32, which serves for receiving signals acquired by a remote medical imaging data collector 10. Server 32 and medical imaging data collector 10 are connected to and/or communicating with each other via a network 20. System 30 according to the present invention further includes an image processor 34. Image processor 34, receives raw data from server 32 and processes it into an image data that is presentable via an image presentation device 36. The image data is also communicable via a network, typically, but not obligatory, the same network, e.g., 20, or a different network 22.

Medical imaging modalities such as digital X-ray (fluoroscopy) modalities, ultrasound devices, magnetic resonance modalities, computerized tomography modalities, positron emission tomography modalities and nuclear medicine modalities, are well known in the art. As is described hereinabove all such modalities include both (i) a “data collector” unit, which functions to scan the patient and collect and convey the signals which result from the scan; and (ii) a data/image processor unit, which is usually a very powerful and expensive computational unit, which receives the raw data/signals that result form the scan and processes them 1o into an image. Thus, in the prior art each imaging modality has it's own computational unit.

As is shown in FIG. 2, medical imaging data collector 10 according to the present invention differs from a corresponding modality in that it includes a “data collector” unit 15, yet does not include a data/image processor unit. Thereby, by itself it is disabled in regard to producing images from data collected thereby. Medical imaging data collector 10 may additionally include, a communication port 11 and a device or a program 13 for compressing, encapsulating and/or tagging outputted raw data signals.

The most trivial way of communicating between collector 10 and system 30 is via high-speed copper lines, using ADSL or VDSL modems. One may use several modems and several telephone lines in parallel. In the latter case, data separating cards/software and data recombining cards/software should be used in collector 10 and system 30, respectively. It will, however, be appreciated that other information transfer, (IT) technologies can be employed, including any fast, wide band, Internet technology, through, for example, cables or satellite.

Examples of such new medical imaging data collectors are provided hereinafter:

A digital X-ray collector (fluoroscopic collector) in which an x-ray source is turned on and x-rays are radiated through a body part of interest. The energy and wavelength of the x-rays allows them to pass through the body part, as the x-rays pass through the hand, for instance, they are attenuated by the different density of tissue they encounter. Bone attenuates a great deal more of the x-rays than soft tissue because of its higher density. It is these differences in absorption that eventually result in an acquired digital signal.

A computerized tomography (CT) data collector is based on the x-ray principal, using a detector that measures the x-ray profile. Inside the covers of a CT scanner there is a rotating frame which has an x-ray tube mounted on one side and a detector mounted on the opposite side. A fan beam of x-ray is created as the rotating frame spins the x-ray tube and detector around the patient. Each time the x-ray tube and detector make a 360° rotation, a “slice” has been acquired. This “slice” is collimated to a thickness between 1 mm and 10 mm using lead shutters in front of the x-ray tube and x-ray detector. As the x-ray tube and detector make a 360° rotation, the detector takes numerous profiles of the attenuated x-ray beam. Typically, in one 360° lap, about 1,000 profiles are sampled. Each profile is subdivided spatially by the detectors and fed into about 700 individual channels. The CT gantry and table have multiple microprocessors that control the rotation of the gantry, movement of the table (up/down and in/out), tilting of the gantry for angled “slices” acquisition, and other functions such as turning the x-ray beam on an off. The CT contains a slip ring that allows electric power to be transferred from a stationary power source onto the continuously rotating gantry. The innovation of the power slip ring has created a renaissance in CT called spiral or helical scanning. These spiral CT scanners can now acquire data from entire anatomic regions like the lungs in a quick 20 to 30 second breath hold. Instead of acquiring a stack of individual slices which may be misaligned due to light patient motion or breathing (and lung/abdomen motion) in between each slice acquisition, spiral CT acquires a volume of data with the patient anatomy all in one position.

A magnetic resonance (MR) data collector is superior to CT in detecting soft tissue lesions such as tumors as it has excellent contrast resolution, meaning it can differentiate subtle soft-tissue changes with exceptional clarity. MR uses magnetic energy and radio waves to acquire data from cross-sectional “slices” of the human body. The main component of most MR systems is a large tube cylindrical shaped magnet. Also, there are MR systems having a C-shaped magnet or other type of open design. The strength of the MR systems magnetic field is measured in metric units called “Tesla”. Most of the cylindrical magnets have a strength between 0.5 and 1.5 Tesla and most of the open or C-shaped magnets have a magnetic strength between 0.01 and 0.35 Tesla. Inside the MR system a magnetic field is created. Each complete MR examination typically includes a series of 2 to 6 sequences. An “MR sequence” is an acquisition of data with specific orientation and a specific “contrast”. During the examination, a radio signal is turned on and off, and subsequently the energy which is absorbed by different atoms in the body is echoed or reflected back out of the body. These echoes are continuously measured by “gradient coils” that are switched on and off to measure the MR signal reflecting back. In the rotating frame of reference, the net magnetization vector rotate from a longitudinal position a distance proportional to the time length of the radio frequency pulse. After a certain length of time, the net magnetization vector rotates 90 degrees and lies in the transverse or x-y plane. It is in this position that the net magnetization can be detected on MRI. The angle that the net magnetization vector rotates is commonly called the ‘flip’ or ‘tip’ angle. At angles greater than or less than 90 degrees there will still be a small component of the magnetization that will be in the x-y plane, and therefore be detected. Radio frequency coils are the “antenna” of the MRI system that broadcasts the RF signal to the patient and/or receives the return signal. RF coils can be “receive-only”, in which case the body coil is used as a transmitter; or transmit and receive (transceiver). Surface coils are the simplest design of coil. They are simply a loop of wire, either circular or rectangular, that is placed over the region of interest. A benefit of MRI is that it can easily acquire direct data from the body in almost any orientation, while CT scanners typically acquire data perpendicular to the long body axis.

An ultrasound data collectors reflect versatile scanning technique all using back reflected sound waves to acquire data from organs or anatomical structures in order to make a diagnosis. The ultrasound process involves placing a device called a transducer, against the skin of the patient near the region of interest, for example, against the back to acquire data from the kidneys. The ultrasound transducer combines functions of emitting and receiving sound. This transducer produces a stream of inaudible, high frequency sound waves which penetrate into the body and echo off the organs inside. The transducer detects sound waves as they echo back from the internal structures and contours of the organs. Different tissues reflect these sound waves differently, yielding a measurable signature. These waves are received by the ultrasound machine and turned into analog or digital signals. Ultrasound scanning has many uses, including, but not limited to, diagnosis of disease and structural abnormalities, helping to conduct other diagnostic procedures, such as needle biopsies, etc.

A nuclear medicine (radioclide scanning) data collectors are designed to collect data pertaining not only of the anatomy of an organ but also to its functionality. This additional acquisition of functional information allows radioclide scanning to help in the diagnosis of certain diseases and medical conditions much sooner than any of the other medical imaging examinations described herein which acquire mainly anatomical data. Nuclear medicine uses radiopharmaceuticals that the target organ preferentially uptakes. These radiopharmaceuticals are injected to or ingested by the patient and than, after a period of time (in which the radiopharmaceuticals is uptake by the organ), radioemission detectors, such as Gamma cameras, are used to detect the radiation emitted from the concentrated radiopharmaceutical and convert it into analog or digital signals (sometimes there is a phase of converting the radioactive signal to light signal and than to an electrical signal).

A positron emission tomography (PET) data collector provides a means for acquiring data pertaining to the rates of biological processes in vivo, which data may be transformed into an image. To this end, two technologies are integrated: (i) a tracer kinetic assay method; and (ii) a computed tomography data collector as is described hereinabove. The tracer kinetic assay method employs a radiolabeled biological active compound (tracer) and the PET data collector measures the tissue tracer concentration. These are supplied to the tracer kinetic model that is a mathematical model that describes the kinetics of the tracer as it participates in a biological process, and permits the calculation of the rate of the process.

Image processor 34 which forms a part of system 30 has the ability to process the signals collected by one or more, preferably all, of the medical image data collectors described hereinabove, and transform them into respective images. The resulting image data varies according to the imaging data collector, of which the original signals are acquired, for example: signal acquired from digital x-ray are processed into a two-dimensional image or set of images of a body part or region; signals acquired from a computerized tomography data collector are presented as “profiles” are backwards reconstructed (or “back projected”) into two-dimensional images of the “slices” that were scanned; in other cases, where spiral or helical CT serve as the origin of the signals, the acquired volume data set can be reconstructed to provide three-dimensional images; when MR data collector is used cross-sectional images or “slices” of the human body are created from the raw data; in the case of an ultrasound data collector the signals are transformed into a two-dimensional image.

System 30 may include one or more image processors, such that, for example, one image processor may have the ability to process data from various types of data collectors; a combination of several image processors each designated to process data from one type of data collector; or system 30 as a whole may be designated to process data from numerous modalities which are all of one or more kind at the same time.

The signal, after being acquired by medical imaging data collector 10, is communicated to system 30 via network 20. This communication may be a simple point-to-point data transfer, however in a presently preferred embodiment of the present invention such communication is effected using data-independent object-oriented encapsulation coding methods.

In the computer industry there has been a shift towards system interoperability through open systems protocols. This shift is being driven by TCP/IP, followed by X-windows (for transmission of windowed graphics), NFS (for file systems access), and new applications level protocols and file formats such as X.500, HTML and SMTP. These protocols and file format standards have allowed interoperability between computers using different operating systems, hardware platforms, and applications suites. Within the Government and industry these data transfer protocols, mostly oriented towards transmission and/or sharing of images and documents, have substantially improved the usefulness of office and home computers. With respect to medical applications, however, such support for multiple platforms or distributed, object-oriented collection and analysis architectures for multiple data types exists to a much lesser degree.

The present invention's preferred embodiment supports general-purpose data routing and encapsulation architecture, which supports input tagging and standardized routing through modem packet switch networks, including the Internet. Important features of the embodiment include: (i) all data are timed and source tagged for later integration into the spatial/temporal reality; (ii) all data are either self descriptive, or encapsulated and object-oriented, so that at any point in the network any software system can acquire data by specific temporal/spatial or content features, and can understand the basic features of the data items; (iii) standard networking models support any reasonable network topology (i.e., support any number of signal acquisition stations delivering data to the image processing system and the image processing system delivering image data to any number of viewing/interpreting stations), and exploit all relevant hardware network implementation standards (ranging from FDDI to RF/Wireless, satellite to land fixed), the network substructure supports geographic distribution of data sources and sinks (i.e., both wide-area and local-area networks); and, (iv) the standards underlying the system are based on public standards and language coding methods for computing system and operating systems independence.

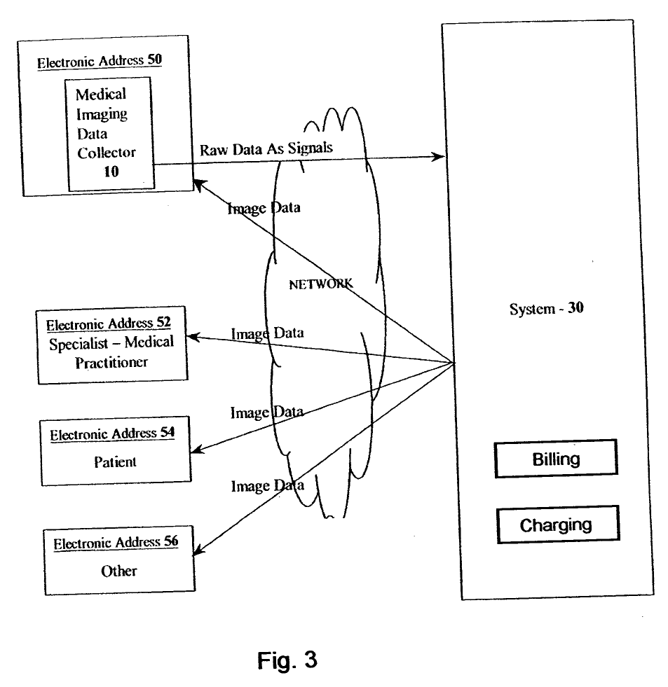

FIG. 3 is a block diagram featuring the data flow to and from system 30 of the present invention. The raw data signals acquired by medical imaging data collector 10 that resides in electronic address 50, are transmitted to system 30 in the manner described hereinabove. System 30, after processing the raw data signals into an image data, communicates the image data for presentation to one or more electronic addresses.

The transmitted image data preferably adheres to the packetisation and encapsulation conventions that were described hereinabove. The image data maybe sent out as a simple datafiles, a pixel data file, a video output signal or as a streaming output signal, depending mainly on the receiving platform, which may be a conventional personal computer (PC).

As mentioned above, after processing the raw data signals into an image, system 30 communicates the image data to one or more electronic addresses, any one of which may serve as a default address to which the image data is transmitted automatically upon completion of it's processing by system 30.

Any one of the following may serve as an electronic address to which the image data may be communicated automatically or upon a request:

First, system 30 may communicate the image data back to the electronic address from which the raw data signal origins which is presented in FIG. 2 as electronic address 50. That means that the image data may be presented via an image presentation device, in real time, at the location where the patient is being examined.

Second, the image data may be further communicated to electronic address 52, which is an address of a medical practitioner or a specialist, for the purpose of analyzing and interpreting the image data.

Analysis and interpretation of images resulting from medical imaging modalities requires extensive skills and experience, not available to any practitioner at any location, specifically in rural or remote areas. In order to overcome these deficiencies, systems have been proposed to communicate acquired and reconstituted medical images to a central location to be analyzed by an experienced personnel, with diagnosis and treatment being relayed back to the remote site. To that effect the reader is referred to, for example, U.S. Pat. Nos. 6,101,407; 5,976,088; 5,949,491; 5,851,186; and 5,482,043; 5,715,823.

In addition, systems were developed to support automated medical information processing for effecting automatic analysis and interpretation of medical diagnostic images. The main system is known as Computer-Aided Diagnosis (CAD). These systems also use Picture Archiving Communication (PAC), which is usually the main storing device for medical diagnostic images. To this end, see U.S. Pat. Nos. 5,807,256; 5,779,634; 5,655,084; 5,644,649 and 5,513,101.

However none of these patents, and no other prior art teaches to physically separate the process of raw data acquisition from image reconstruction, as is offered by the present invention.

Third, the image data may naturally be communicated to an electronic address 54 of the patient himself for self documentary.

Fourth, system 30 may also communicate the image data to any other electronic address 56. These other electronic addresses may be of a consultant, an insurance company, different clinics or hospitals, etc.

Any communication of image data to any one of the above mentioned addresses is done only upon authorization by the patient and/or the paying client, with compliance to medical secrecy and other legal issues.

As is further shown in FIG. 1, system 30 preferably further includes and/or communicating with a data storage device 38. Data storage device 38 may be any device comprising a database that supports the storage of image data coupled with storing any other data that describes and/or defines and/or is otherwise pertaining to the image data, such as: patient's details, description of the image data, details of the data collector modality, analysis of the image data, diagnosis, details of health center, details of medical insurance company, etc. Data storage device 38 also supports the retrieval of any image data which is stored therein, according to it's defining data as is described above.

System 30 may be further constructed so as to be able to perform automatic analysis and/or interpretation of the image data, which image data may be a newly reconstructed image data or image data retrieved from data storage device 38. U.S. Pat. Nos. 6,101,407; 5,976,088; 5,949,491; 5,851,186; and 5,482,043; 5,715,823 teach automatic analysis and/or interpretation of the image data and are therefore incorporated herein by reference.

System 30 may be further designed to supply billing services and effect secure credit/debit transactions, so that a client may order a variety of services to be performed by system 30, and may be effectively billed and charged for services rendered thereto. Such services may include any one or a combination of the following: performing a scan of a patient at a remote location, processing the resulting signals into an image data, automatically analyzing the image data and sending the analysis report to the client, storing the image data, retrieval of the image data and communicating the retrieved image to the client or anyone on his behalf, supplying a client with an analysis and interpretation of an image data made electronically or by a specialist, etc.

Any one of the following may constitute a client, depending upon proper consent of the imaged patient in accordance with patient confidentiality and legal limitations: the patient himself, a treating medical practitioner, an insurance company, an HMO service, a healthcare facility, etc.

Thus, in medical imaging, the system of the present invention allows for separating the technologies which are actually used to scan a patient and acquire raw imagery data. (which is transformed into an electronic analog or digital signal) from the technologies that are used to process that data, transform it into an image and manipulate it.

According to the present invention the actual image processing of raw data signals from any one of a plurality of image data collectors is performed at and by a central server, obviating the need for installing expensive computing hardware and software to each collector so as to enable in situ image reconstitution as is solely done today. This, in turn, will reduce the costs of medical imaging and will increase their affordability and use. It will also increase the ability of data sharing with respect to medical images as has been the case in other fields, all of which will result in better public health.

It will be appreciated that the present invention may take advantage of recently developed technologies and in the future of technologies to be developed which relate to data packetisation, wide band communication, especially with respect to the Internet, data compression and decompression and automatic image, reconstitution, analysis and interpretation.

The current state of technology in these fields is sufficient to provide the system of the present invention with high performance in terms of time of image reconstitution, superior over what is presently offered by existing imaging modalities, since the system of the present invention offers sharing of expensive resources, which enables the usage of far superior and expensive computation to be exercised.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for remotely producing a diagnostic image, the system comprising:
(a) a server designed and constructed for receiving, via a first communications network, at least one signal acquired by a remote imaging data collector; and
(c) an image processor for processing said at least one signal into image data being communicable via a second communications network and presentable via an image presentation device.

2. The system of claim 1, wherein said server is further designed and constructed for automatically communicating said image data back to a default electronic address.

3. The system of claim 2, wherein said default electronic address is received by said server, via said first communications network, along with said at least one signal.

4. The system of claim 1, wherein said first communications network and said second communications network are a single communications network.

5. The system of claim 1, wherein said imaging data collector is selected from the group consisting of a digital X-ray data collector, an ultrasound data collector, a magnetic resonance modality data collector, a computerized tomography data collector, a positron emission tomography data collector and a nuclear medicine data collector.

6. The system of claim 1, wherein said at least one signal is selected from the group consisting of a raw digital signal, a raw analog signal, and a raw analog signal converted into a digital signal.

7. The system of claim 1, wherein each of said at least one signal is encapsulated, including the packetisation of each of said at least one signal so as to be self descriptive.

8. The system of claim 1, wherein said image data is encapsulated, including the packetisation of said image data so as to be self descriptive.

9. The system of claim 1, wherein said image data is represented as a file containing pixel data.

10. The system of claim 1, wherein said image data is represented as a video output signal.

11. The system of claim 1, wherein said image data is represented as a streaming output signal.

12. The system of claim 1, wherein said image data represents a two dimensional image.

13. The system of claim 1, wherein said image data represents a three dimensional image.

14. The system of claim 1, further comprising a data storage device for allowing storage and later retrieval of said image data.

15. The system of claim 1, wherein said server is further designed and constructed for automatically analyzing said image data.

16. The system of claim 1, wherein said server is further designed and constructed for automatically interpreting said image data.

17. A method for remotely producing a diagnostic image, the method comprising the steps of:
(a) at a source location, using an imaging data collector for acquiring at least one signal;
(b) communicating said at least one signal via a first communications network to a remote location;
(c) at said remote location, processing said at least one signal into image data; and
(d) communicating said image data via a second communications network to said source location or to a third location.

18. The method of claim 17, wherein said first communications network and said second communications network are a single communications network.

19. The method of claim 17, wherein said imaging data collector is selected from the group consisting of a digital X-ray data collector, an ultrasound data collector, a magnetic resonance modality data collector, a computerized tomography data collector, a positron emission tomography data collector and a nuclear medicine data collector.

20. The method of claim 17, wherein said at least one signal is selected from the group consisting of a raw digital signal, a raw analog signal, and a raw analog signal converted into a digital signal.

21. The method of claim 17, further comprising the steps of:
(a) encapsulating said at least one signal; and
(b) packeting of said at least one signal so as to be self descriptive.

22. The method of claim 17, further comprising the steps of:
(a) encapsulating said image data; and
(b) packeting said image data so as to be self descriptive.

23. The method of claim 17, further comprising the step of representing said image data as a file containing pixel data.

24. The method of claim 17, further comprising the step of representing said image data as a video output signal.

25. The method of claim 17, further comprising the step of representing said image data as a streaming output signal.

26. The method of claim 17, wherein said image data represents a two dimensional image.

27. The method of claim 17, wherein said image data represents a three dimensional image.

28. The method of claim 17, further comprising the step of storing said image data for later retrieval.

29. The method of claim 17, further comprising the step of automatically analyzing said image data.

30. The method of claim 17, further comprising the step of automatically interpreting said image data.

31. A medical diagnostic device comprising an imaging data collector and lacking image producing capability.

32. The device of claim 31, further comprising a communication port, the device being designed and constructed for communicating raw data collected thereby to a remote location.

33. A medical diagnostic device consisting essentially of at least one sensor for collecting raw data being processable into an image, and a communication port for communicating said raw data to a remote location.

34. A system for providing image reconstitution services, the system comprising:
(a) a server designed and constructed for receiving, via a first communications network, at least one signal acquired by a remote imaging data collector; and
(b) an image processor for processing said at least one signal into image data being communicable via a second communications network and presentable via an image presentation device.

35. The system of claim 34, wherein said first communications network and said second communications network are a single communications network.

36. The system of claim 34, wherein said server being further designed and constructed for billing a client.

37. The system of claim 34, wherein said server is further designed and constructed for effecting secure credit/debit transactions.

38. The system of claim 34, wherein said imaging data collector is selected from the group consisting of a digital X-ray data collector, an ultrasound data collector, a magnetic resonance data collector, a computerized tomography data collector, a positron emission tomography data collector and a nuclear medicine data collector.

39. The system of claim 34, wherein said at least one signal is selected from the group consisting of a raw digital signal, a raw analog signal, and a raw analog signal converted into a digital signal.

40. The-system of claim 34, wherein said at least one signal is encapsulated, including the packetisation of said at least one signal so as to be self descriptive.

41. The system of claim 34, wherein said image data is encapsulated, including the packetisation of said image data so as to be self descriptive.

42. The system of claim 34, wherein said image data is represented as a file containing pixel data.

43. The system of claim 34, wherein said image data is represented as a video output signal.

44. The system of claim 34, wherein said image data is represented as a streaming output signal.

45. The system of claim 34, wherein said image data represents a two dimensional image.

46. The system of claim 34, wherein said image data represents a three dimensional image.

47. The system of claim 34, further comprising a data storage device for effecting storage of said image data and retrieval of said image data.

48. The system of claim 34, wherein said server is further designed and constructed for automatically communicating said image data back to a default electronic address.

49. The system of claim 48, wherein said default electronic address is received by said server, via said first communications network, along with said at least one signal.

50. The system of claim 34, wherein said image data is sent, via said second communications network, to an electronic address of a healthcare practitioner, said practitioner being skilled at analyzing and interpreting said image data.

51. The system of claim 34, wherein said image data is sent, via said second communications network, to an electronic address of a patient.

52. The system of claim 34, wherein said image data is sent, via said second communications network, to any electronic address desirable and authorized by any one of a group comprising the patient, a Health Management Organization, a healthcare facility, an insurance company and any combination thereof.

53. The system of claim 34, wherein said server is further designed and constructed for automatically analyzing said image data.

54. The system of claim 34, wherein said server is further designed and constructed for automatically interpretation of said image data.

55. The system of claim 43, wherein said image data is retrieved from said data storage device and sent, via said communications network, to an electronic address of a healthcare practitioner, said practitioner being skilled at analyzing and interpreting said image data.

56. The system of claim 43, wherein said image data is retrieved from said data storage device and sent, via said communications network, to an electronic address of a patient.

57. The system of claim 47, wherein said image data is retrieved from said a data storage device and sent, via said communications network, to any electronic address desirable and authorized by any one of a group comprising the patient, a Health Management Organization, a healthcare facility, an insurance company and any combination thereof.

58. The system of claim 47, wherein said server is further designed and constructed for automatically analyzing said retrieved image data.

59. The system of claim 47, wherein said server is further designed and constructed for automatically interpreting said retrieved image data.

60. A method for providing image reconstitution services, the method comprising the steps of:
(a) receiving at least one signal acquired by a remote imaging collector via a first communications network; and
(b) processing said at least one signal into image data, said image data being presentable via an image presentation device; and
(c) communicating said image data via a second communications network.

61. The method of claim 60, wherein said first communications network and said second communications network are a single communications network.

62. The method of claim 60, further comprising the step of:
automatically communicating said image data back to a default electronic address.

63. The method of claim 60, wherein said default electronic address is received, via said first communications network, along with said at least one signal.

64. The method of claim 60, further comprising the step of:
automatically billing the client.

65. The method of claim 60, further comprising the step of:
automatically effecting secure credit/debit transactions.

66. The method of claim 60, wherein said imaging data collector is selected from the group consisting of a digital X-ray data collector, an ultrasound data collector, a magnetic resonance modality data collector, a computerized tomography data collector, a positron emission tomography data collector and a nuclear medicine data collector.

67. The method of claim 60, wherein said at least one signal is selected from the group consisting of a raw digital signal, a raw analog signal, and a raw analog signal converted into a digital signal.

68. The method of claim 60, further comprising the steps of:
(a) encapsulating said at least one signal and said data; and
(b) packeting said at least one signal and said data so as to be self descriptive.

69. The method of claim 60, further comprising the steps of:
(a) encapsulating said image data; and
(b) packeting said image data so as to be self descriptive.

70. The method of claim 60, further comprising the step of:
representing said image data as a file containing pixel data.

71. The method of claim 60, further comprising the step of:
representing said image data as a video output signal.

72. The method of claim 60, further comprising the step of:
representing said image data as a streaming output signal.

73. The method of claim 60, wherein said image data represents a two dimensional image.

74. The method of claim 60, wherein said image data represents a three dimensional image.

75. The method of claim 60, further comprising the steps of:
(a) storing said image data in a data storage device; and
(b) retrieving said image data from a data storage device.

76. The method of claim 60, further comprising the step of sending said image data, via said second communications network, to an electronic address of a healthcare practitioner, said practitioner being skilled at analyzing and interpreting said image data.

77. The method of claim 60, further comprising the step of sending said image data, via said second communications network, to an electronic address of a patient.

78. The method of claim 60, further comprising the step of sending said image data, via said second communications network, to any electronic address desirable and authorized by any one of a group comprising the patient, a Health Management Organization, a healthcare facility, an insurance company and any combination thereof.

79. The method of claim 60, further comprising the step of providing a service of automatically analyzing said image data.

80. The method of claim 60, further comprising the step of providing a service of automatically interpreting said image data.

81. The method of claim 75, further comprising the steps of:
(a) retrieving said image data from said data storage device; and
(b) sending said image data, via said communications network, to an electronic address of a healthcare practitioner, said practitioner being skilled at analyzing and interpreting said image data.

82. The method of claim 75, further comprising the steps of:
(a) retrieving said image data from said a data storage device; and
(b) sending said image data, via said communications network, to an electronic address of a patient.

83. The method of claim 75, further comprising the steps of:
(a) retrieving said image data from said a data storage device; and
(b) sending said image data, via said communications network, to any electronic address desirable and authorized by any one of a group comprising the patient, a Health Management Organization, a healthcare facility, an insurance company and any combination thereof.

84. The method of claim 75, further comprising the steps of:
automatically analyzing said retrieved image data.

85. The method of claim 75, further comprising the steps of:
automatically interpreting said retrieved image data.
